(19) United States
(12) Patent Application Publication
(10) Pub. No.: US 2018/0016217 A1
(43) Pub. Date: Jan. 18, 2018

(54) METHODS FOR PREPARING GLYCEROL TERT-BUTYL ETHERS

(71) Applicant: SABIC Global Technologies, B.V., Bergen op Zoom (NL)

(72) Inventors: Sunil Shivajirao Dhumal, Maharashtra (IN); Vinod Sankaran Nair, Bangalore (IN); Rekha Mahadevaiah, Bangalore (IN); Umesh Krishna Hasyagar, Bangalore (IN)

(73) Assignee: SABIC Global Technologies, B.V., Bergen Op Zoom (NL)

(21) Appl. No.: 15/542,981

(22) PCT Filed: Jan. 28, 2016
(86) PCT No.: PCT/IB2016/050444

§ 371 Date: Jul. 12, 2017

Related U.S. Application Data
(60) Provisional application No. 62/110,152, filed on Jan. 30, 2015.

(51) Int. Cl. C07C 41/06

(52) U.S. Cl. CPC C07C 41/06

(57) ABSTRACT

Methods for generating glycerol tert-butyl ethers include reacting isobutylene and glycerol in the presence of a catalyst and a co-solvent cum co-reactant to produce a mono-, di-, or tri-glycerol tert-butyl ether, where the isobutylene and glycerol are partially miscible.

### FIELD

This invention relates to methods for generating glycerol tert-butyl ethers.

### BACKGROUND

Glycerol, also known as glycerin or glycerine, is a colorless, odorless, viscous liquid. Glycerol has various uses, including, for example, as an antifreeze agent and as an excipient in certain pharmaceutical preparations. Glycerol occurs in nature and can also be prepared synthetically by various routes. Among the sources of glycerol is the commercial production of biodiesel. Because glycerol is a triol and has three hydroxyl groups, glycerol is capable of derivatization to provide glycerol ethers that include mono-, di- and triether compounds.

Glycerol ethers are ether compounds where at least one of the carbon moieties attached to an ether linkage is derived from glycerol. Glycerol ethers can be derived from renewable sources and are of interest as a renewable source of energy. Examples of glycerol ethers include glycerol tert-butyl ethers (GTBEs). Five structurally distinct glycerol tert-butyl ethers can be formed. The first two compounds, 1-tert-butyl glycerol and 2-tert-butyl glycerol, are mono-tert-butyl glycerol ethers (mono-GTBEs, also known as m-GTBEs). The next two compounds, 1,3-di-tert-butyl glycerol and 1,2-di-tert-butyl glycerol, are di-tert-butyl glycerol ethers (di-GTBEs). The last compound, tri-tert-butyl glycerol, is tri-tert-butyl glycerol ether (tri-GTBE, also known as t-GTBE). Di-GTBEs and tri-GTBEs can sometimes be known as higher GTBEs or h-GTBEs.

GTBEs can have useful properties as fuel additives. For example, methyl tert-butyl ether (MTBE) is soluble in diesel, biodiesel and other fuels and is a commonly used oxygenate fuel additive. Like MTBE, GTBEs can be soluble in diesel, biodiesel and other fuels and can be used as oxygenate fuel additives. For example, di-GTBEs and tri-GTBE are desirable as fuel additives, as they have good solubility in diesel and biodiesel fuels, as compared to MTBE.

Certain existing methods of generating GTBEs are known in the art. U.S. Patent Application No. 2011/0098510 discloses a process for the production of GTBE that includes catalyzed etherification of glycerol and isobutylene in a multi-phase system followed by neutralization of the acid catalyst used in the etherification reaction and removal of salts from the reaction. PCT Patent Application No. WO 2009/066925 discloses a process for the production of GTBE from glycerol and isobutylene in the presence of a dioxane co-solvent. Korean Patent No. KR101104570 discloses a process for the production of GTBE from glycerol and isobutylene by an etherification reaction in the presence of a solvent. U.S. Patent Application No. 2013/0031829 discloses a process for the production of GTBE that includes the extraction of the glycerol ether from the reaction mixture using a solvent.

Etherification reactions of glycerol with isobutylene under standard conditions can suffer from certain drawbacks. For example, etherification of glycerol with isobutylene can suffer from mass transfer limitations caused by a non-optimal contact between isobutylene and glycerol liquid phases. Other drawbacks to etherification of glycerol with isobutylene can include undesired secondary reactions (e.g., oligomerization of isobutylene), poor control of product selectivity (e.g., poor control of the relative output of m-GTBEs, di-GTBEs, and tri-GTBE) and limited catalyst lifetime.

Thus, there remains a need in the art for improved methods for generating glycerol tert-butyl ethers.

### SUMMARY

The presently disclosed subject matter provides methods for generating glycerol tert-butyl ethers.

In certain embodiments, a method for generating a glycerol tert-butyl ether includes reacting glycerol and isobutylene in the presence of a catalyst and a co-solvent cum co-reactant to generate a mono-, di-, or tri-glycerol tert-butyl ether, where the isobutylene and glycerol are partially miscible. In certain embodiments, the co-solvent cum co-reactant can include methanol, ethanol, methyl tert-butyl ether, ethyl tert-butyl ether, or a combination comprising at least one of the foregoing.

In certain embodiments, the method can further include separating the di- and tri-glycerol tert-butyl from the mono-glycerol tert-butyl ether and recycling the co-solvent cum co-reactant, unreacted glycerol, if any, and mono-glycerol tert-butyl for further generation of a mono-, di-, or tri-glycerol tert-butyl ether product.

In certain embodiments, the co-solvent cum co-reactant is methanol. In certain embodiments, the method can further include reacting methanol with isobutylene to produce methyl tert-butyl ether. In certain embodiments, the method can further include separating the di- and tri-glycerol tert-butyl and methyl tert-butyl ether from the mono-glycerol tert-butyl ether and recycling the co-solvent cum co-reactant, unreacted glycerol, if any, and mono-glycerol tert-butyl for further generation of a mono-, di-, or tri-glycerol tert-butyl ether product.

In certain embodiments, the co-solvent cum co-reactant is ethanol. In certain embodiments, the method can further include reacting ethanol with isobutylene to produce ethyl tert-butyl ether. In certain embodiments, the method can further include separating the di- and tri-glycerol tert-butyl ether and ethyl tert-butyl ether from the products and recycling the co-solvent cum co-reactant, unreacted glycerol, if any, and mono-glycerol tert-butyl for further generation of mono-, di-, or tri-glycerol tert-butyl ether products.

In certain embodiments, the catalyst is an acid catalyst. In certain embodiments, the acid catalyst can include sulfuric acid, p-toluene sulfonic acid, methanesulfonic acid, or a combination comprising at least one of the foregoing.

In certain embodiments, a method for the production of glycerol tert-butyl ether can include reacting isobutylene and glycerol in the presence of a catalyst and a co-solvent cum co-reactant including methanol, ethanol, methyl tert-butyl ether, ethyl tert-butyl ether, or a combination comprising at least one of the foregoing to produce glycerol tert-butyl, where the isobutylene and glycerol are partially miscible.

In certain embodiments, a method for the production of glycerol tert-butyl ether can include reacting isobutylene and glycerol in the presence of a catalyst and a co-solvent including methanol, methyl tert-butyl ether and mono-glycerol tert-butyl ether to produce a mono-, di-, or tri-glycerol tert-butyl ether, where the isobutylene and glycerol are partially miscible.

In certain embodiments, a method for the production of glycerol tert-butyl ether and methyl tert-butyl ether can include reacting isobutylene and glycerol in the presence of a catalyst and methanol to produce a mono-, di-, or tri-glycerol tert-butyl ether and methyl tert-butyl ether, where the isobutylene and glycerol are partially miscible.

In certain embodiments, a method for the production of glycerol tert-butyl ether and ethyl tert-butyl ether can include reacting isobutylene and glycerol in the presence of a catalyst and ethanol to produce a mono-, di-, or tri-glycerol tert-butyl ether and ethyl tert-butyl ether, where the isobutylene and glycerol are partially miscible.

### BRIEF DESCRIPTION OF THE DRAWINGS

FIG. 1 is a schematic diagram showing an exemplary method for generating glycerol tert-butyl ether in accordance with a non-limiting embodiment of the disclosed subject matter.

FIG. 2 is a schematic diagram showing an exemplary method for generating glycerol tert-butyl ether and methyl tert-butyl ether in accordance with a non-limiting embodiment of the disclosed subject matter.

FIG. 3 is a schematic diagram showing an exemplary method for generating glycerol tert-butyl ether and ethyl tert-butyl ether in accordance with a non-limiting embodiment of the disclosed subject matter.

FIG. 4 is a schematic diagram showing an exemplary method for generating glycerol tert-butyl ether in accordance with a non-limiting embodiment of the disclosed subject matter.

FIG. 5 is a schematic diagram showing an exemplary method for generating glycerol tert-butyl ether in accordance with a non-limiting embodiment of the disclosed subject matter.

### DETAILED DESCRIPTION

Described herein are methods for generating glycerol tert-butyl ethers (GTBEs) from glycerol and isobutylene.

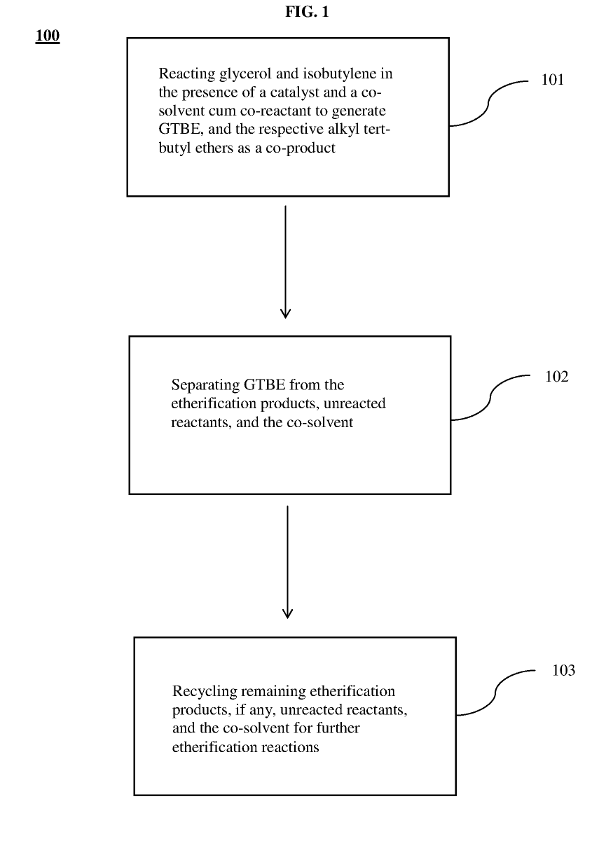

For the purpose of illustration and not limitation, FIG. 1 is a schematic representation of an exemplary method according to the disclosed subject matter. In certain embodiments, the presently disclosed method 100 can include the production of GTBE, along with the respective alkyl tert-butyl ether as a co-product, from glycerol and iso-butylene in the presence of one or more co-solvents (e.g., co-solvent cum co-reactant) and a catalyst 101.

In certain embodiments, the glycerol for use in producing GTBE can be high purity glycerol. For example, and not by way of limitation, the glycerol can be from 95% to 100% pure. In certain embodiments, the glycerol for use in the presently disclosed methods can be produced as a byproduct in the production of biodiesel.

Isobutylene is a simple olefin (alkene) compound also known as 2-methylpropene, isobutene, gamma-butylene, 2-methylpropylene, methylpropene, and 2-methyl-1-propene. The isobutylene for use in the disclosed method can be high purity isobutylene. For example, and not by way of limitation, the isobutylene can be from 70% to 100% pure. In certain embodiments, the isobutylene can be from 75% to 100%, from 80% to 100%, from 85% to 100%, from 90% to 100%, from 95% to 100% pure.

The etherification reactions can be conducted at a temperature and a pressure at which isobutylene and glycerol are in liquid phases. In certain embodiments, the etherification reactions can be conducted at temperatures and pressures known in the art. For example, in certain non-limiting embodiments, the reactions can be conducted at a temperature between 60° C. and 90° C. and at a pressure between 8 bar and 20 bar.

As noted above, the etherification reaction of isobutylene and glycerol can include the use of a co-solvent. Because glycerol and isobutylene are immiscible, etherification reactions of glycerol and isobutylene can involve two distinct liquid phases: a relatively polar phase rich in glycerol and a relatively nonpolar phase rich in isobutylene. There is some isobutylene dissolved in the glycerine phase and most of the etherification reaction occurs in this phase. Mass transfer from the isobutylene phase to the glycerine phase maintains the supply of isobutylene in the glycerine phase. However, the existence of two substantially distinct, immiscible liquid phases can result in reduced contact between the phases, which can limit mass transfer. Limited mass transfer can, in turn, reduce the reaction rate and can, in some cases, accelerate undesired side reactions. Non-limiting examples of undesired side reactions in etherification reactions of glycerol and isobutylene can include oligomerization of isobutylene, disproportionation reactions of the glycerol ether products (GTBEs) and decomposition reactions of the GTBEs. In addition, glycerol is highly viscous, which can make reactant or reaction mixture transfer and mixing more difficult, and therefore more costly.

The addition of a co-solvent (e.g., a co-solvent cum co-reactant) to the etherification reaction of glycerol and isobutylene can increase the miscibility between the glycerol and isobutylene to result in a more homogenous single liquid phase, and can improve mass transfer, improve reaction rate, and reduce side reactions. For example, and not by way of limitation, the inclusion of a co-solvent in the etherification reaction can result in the isobutylene and glycerol becoming partially miscible. In certain embodiments, “partially miscible” can refer to the presence of less than or equal to 10%, less than or equal to 5% of isobutylene within the glycerol phase, or less than or equal to 10%, less than or equal to 5% of glycerol within the isobutylene phase. In an embodiment, “partially miscible” can refer to 2 mole % to less than or equal to 10 mole %, or greater than 2 mole % to less than or equal to 5 mole % of isobutylene within the glycerol phase under stoichiometric reaction conditions (molar ratio of isobutylene to glycerol=3:1), or 3 to less than or equal to 10 mole %, or greater than 3 to less than or equal to 5 mole % of glycerol within the isobutylene phase under stoichiometric reaction conditions (molar ratio of isobutylene to glycerol=3:1). In certain embodiments, a GTBE synthesis method at 80° C. and 16 bar pressure that includes 20 mole % of alkyl tert-butyl ether can have the following miscibility pattern predicted from Aspen-based calculations. The isobutylene miscibility in glycerol can be 3 mole % of isobutylene in glycerol with respect to the overall glycerol phase. In certain embodiments, glycerol miscibility in isobutylene can be 2 mole % with respect to the overall isobutylene phase.

In certain embodiments, the co-solvent can be a compound that increases the miscibility of glycerol and isobutylene or the etherification products such as mono-, di-, and tri-GTBEs. In certain embodiments, the solvent can also function as a co-reactant (i.e., a co-solvent cum co-reactant) to produce additional etherification products, e.g., methyl tert-butyl ether (MTBE) or ethyl tert-butyl ether (ETBE). For example, and not by way of limitation, the solvent can be an ether, such as mono-tert-butyl glycerol ether (m-GTBE), methyl tert-butyl ether (MTBE), ethyl tert-butyl ether (ETBE), methanol, ethanol, or a combination comprising at least one of the foregoing. In certain embodiments, the co-solvent cum co-reactant is methanol. In certain embodiments, the co-solvent cum co-reactant is ethanol.

The co-solvent used in the presently disclosed methods can be added to the reaction as a mixture with glycerol. For example, and not by way of limitation, the co-solvent can be pre-mixed with glycerol and added with the glycerol feedstream into a reactor configured to perform an etherification reaction, as described below. Alternatively or additionally, the co-solvent can be added to the reaction separately from the glycerol.

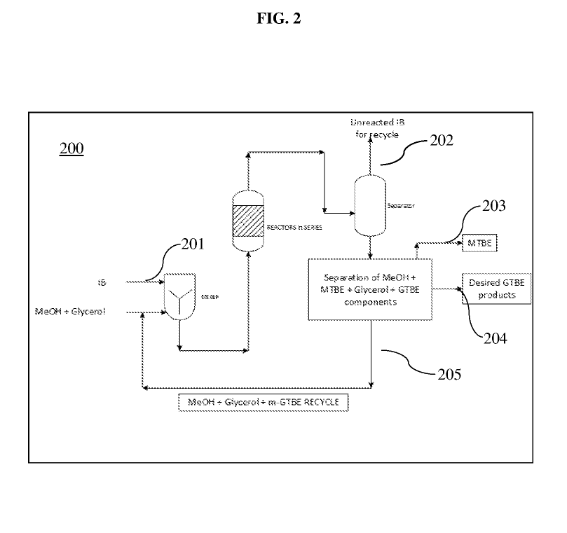

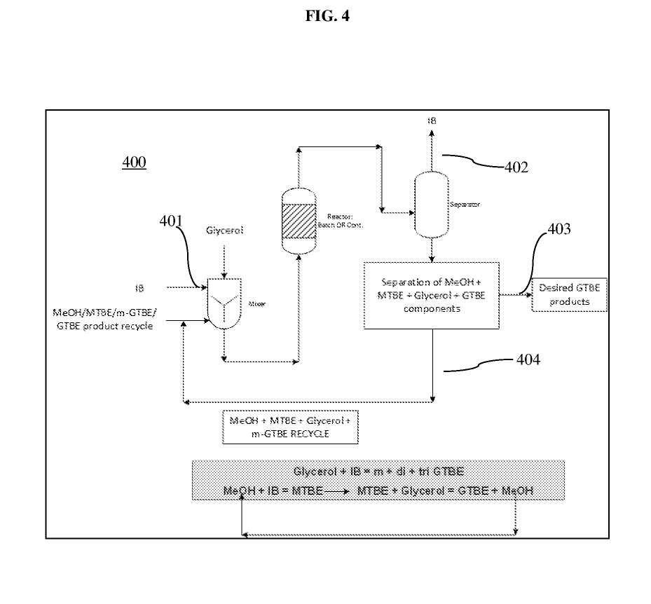

In certain embodiments, methanol can be used as a co-solvent cum co-reactant for the GTBE etherification reaction of isobutylene and glycerol. As embodied herein and depicted in FIG. 2, in certain embodiments, methanol can be used to increase the miscibility of the glycerol and isobutene phases and further react with isobutylene to produce MTBE as an etherification product. In certain embodiments, the MTBE generated by the etherification of isobutylene and methanol can further react with the glycerol within the reactants to generate GTBE and methanol (see FIG. 4). Without being bound by theory, it is believed that the production of MTBE improves the production of GTBE by improving the flow properties of the reaction mixtures, and by improving the reaction kinetics of producing GTBE.

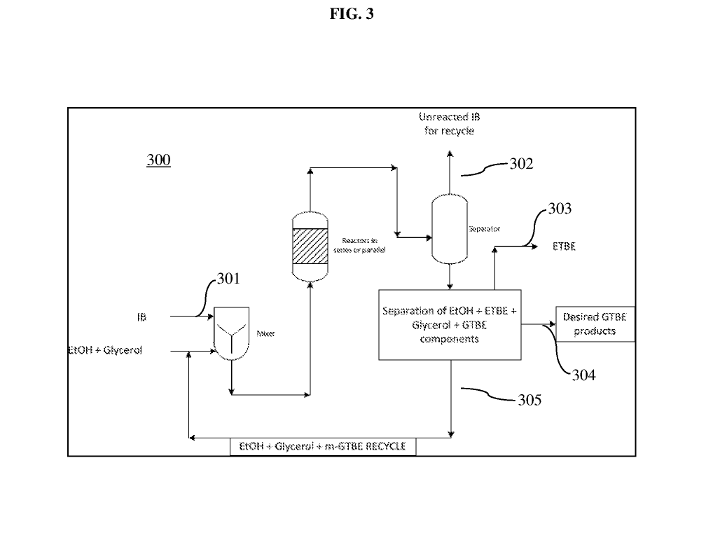

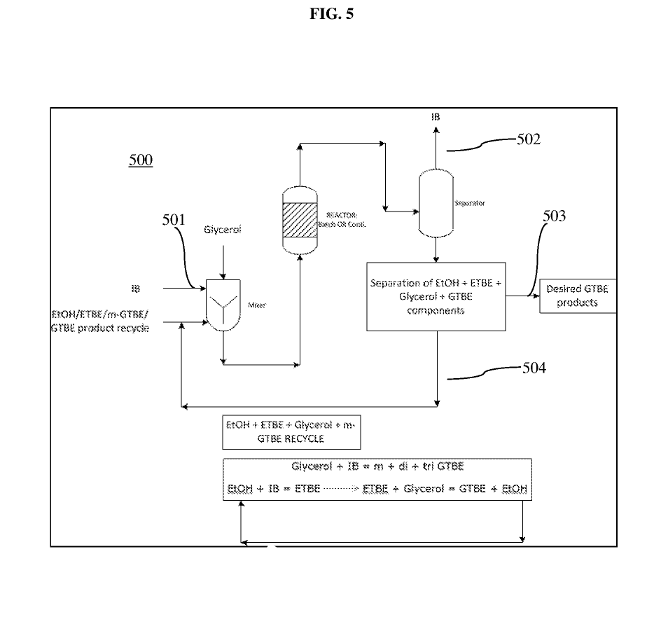

In certain embodiments, ethanol can be used as a co-solvent cum co-reactant for the GTBE etherification reaction of isobutylene and glycerol. As embodied herein and depicted in FIG. 3, in certain embodiments, ethanol can be used to increase the miscibility of the glycerol and isobutene phases and co-react with isobutylene to produce ETBE as an etherification product. In certain embodiments, the ETBE generated by the etherification of isobutylene and ethanol can further react with the glycerol within the reactants to generate GTBE and ethanol (see FIG. 5). Without being bound by theory, it is believed that the production of MTBE improves the production of GTBE by improving the flow properties of the reaction mixtures, and by increasing the reaction kinetics of producing GTBE.

The etherification reaction of glycerol and isobutylene can be performed in the presence of 5% to 50% by weight of the co-solvent cum co-reactant, as compared to the weight of glycerol. For example, and not by way of limitation, the etherification reaction can occur in the presence of 5% to 45%, or 5% to 40%, or 5% to 35%, or 5% to 30%, or 5% to 25%, or 5% to 20%, or 5% to 15%, or 5% to 10%, or 10% to 50%, or 15% to 50%, or 20% to 50%, or 25% to 50%, or 30% to 50%, or 35% to 50%, or 40% to 50%, or 45% to 50% by weight of the co-solvent cum co-reactant, as compared to the weight of glycerol.

The catalyst for use in the presently disclosed subject matter can include any catalyst known in the art that can catalyze the etherification of glycerol and isobutylene. The catalyst can be a homogenous catalyst or a heterogeneous catalyst, or a combination comprising at least one of the foregoing. In certain embodiments, the catalyst can be an acid catalyst such as, but not limited to, a Bronsted acid, a Lewis acid or a Bronsted-Lowry catalyst. Non-limiting examples of suitable catalysts for the etherification of glycerol and isobutylene to generate GTBEs include, but are not limited to, molecular sieves (e.g., 4A molecular sieves), ion-exchange resins (e.g., AMBERLYST® resins and AMBERLITE® resins), sulfuric acid, acetic acid, formic acid, hydrochloric acid, sulfamic acid, methanesulfonic acid, phosphoric acid, p-toluene sulfonic acid, heteropoly acids (HPAs), ionic liquids, solid acid catalysts, and trifluoroacetic acid. In certain embodiments, the ion-exchange resin can be generated through a substitution reaction of a metal ion and an ion-exchange resin. For example, and not by way of limitation, the metal capable of being substituted with the ion-exchange resin includes Li, Na, Ba, Mg, Cs, Al, La, Ag, or a combination comprising at least one of the foregoing.

Heterogeneous catalysts can be used as solid particulates dispersed in a liquid phase, or heterogeneous catalysts can be immobilized on a solid support. Non-limiting examples of solid supports used to prepare heterogeneous catalysts can include various metal salts, metalloid oxides, and metal oxides, e.g., titanium oxide, zirconium oxide, silica (silicon oxide), alumina (aluminum oxide), magnesium oxide, and magnesium chloride. The catalyst used in the present disclosure can be of any shape and size. For example, but not by way of limitation, the catalyst can be in the form of powder, granules, spheres, pellets, beads, cylinders, trilobe, and quadralobe shaped pieces.

The etherification reaction of glycerol and isobutylene can be performed with a catalyst loading of 2% to 10%, by weight, as compared to the weight of glycerol. For example, and not by way of limitation, the etherification reaction can be performed with a catalyst loading of 2% to 9%, or 2% to 8%, or 2% to 7%, or 2% to 6%, or 2% to 5%, or 2% to 4%, or 2% to 3%, or 3% to 10%, or 4% to 10%, or 5% to 10%, or 6% to 10%, or 7% to 10%, or 8% to 10%, or 9% to 10%, or 3% to 8%, or 3% to 7%, or 4% to 8%.

The methods of the presently disclosed subject matter can further include the separation of the desired ethers (e.g., di-GTBEs o tri-GTBEs) from the etherification products 102. In certain embodiments, the methods can further include the recycling of the co-solvent, unreacted reactants, e.g., glycerol, and mono-GTBE for use as the solvent and as the reactants in further etherification reactions 103. In certain embodiments, unreacted isobutylene can be separated from the co-solvent, glycerol, and etherification products prior to the recycling step or separation step.

As embodied herein and depicted in FIGS. 2 and 4, the presently disclosed methods can include the etherification of isobutylene and glycerol in the presence of methanol as a co-solvent cum co-reactant to produce the etherification products, GTBE or MTBE 201 or 401. In certain embodiments, the desired GTBEs, e.g., di-GTBEs and tri-GTBEs, can be separated from the other etherification products, co-solvent, and unreacted reactants to produce a GTBE product stream 204 or 403, and the co-solvent, unreacted reactants, e.g., glycerol, and m-GTBE can be recycled for use as the solvent or as the reactants in further etherification reactions 205. In certain embodiments, MTBE can be separated from the other etherification products, co-solvent, and unreacted reactants to produce a MTBE product stream 203. Alternatively or additionally, and in reference to FIG. 4, the MTBE can be recycled with the co-solvent, unreacted reactants, and m-GTBE to be used as a solvent or reactant in further etherification reactions 404. In certain embodiments, the MTBE can be partially separated and partially recycled. In certain embodiments, “partially separated” can include the separation of 5%, 10%, 20%, 30%, 40%, 50%, 60%, 70% or more of the ether by weight. In certain embodiments, “partially recycled” can include the recycling of 5%, 10%, 20%, 30%, 40%, 50%, 60%, 70% or more of the ether by weight. In certain embodiments, unreacted isobutylene can be separated from the co-solvent, glycerol, and etherification products prior to the recycling step or separation step 202 or 402.

As embodied herein and depicted in FIGS. 3 and 5, the presently disclosed methods can include the etherification of isobutylene and glycerol in the presence of ethanol as a co-solvent cum co-reactant to produce the etherification products, GTBE, or ETBE 301 or 501. In certain embodiments, the desired GTBEs, e.g., di-GTBEs and tri-GTBEs, can be separated from the other etherification products, co-solvent, and unreacted reactants to produce a GTBE product stream 304 or 503, and the co-solvent, unreacted reactants, e.g., glycerol, and m-GTBE can be recycled for use as the solvent or as the reactants in further etherification reactions 305. In certain embodiments, ETBE can be separated from the other etherification products, co-solvent, and unreacted reactants to produce an ETBE product stream 303. Alternatively or additionally, and in reference to FIG. 5, the ETBE can be recycled with the co-solvent, unreacted reactants, and m-GTBE to be used as a solvent or reactant in further etherification reactions 504. In certain embodiments, the ETBE can be partially separated and partially recycled, as described above. In certain embodiments, unreacted isobutylene can be separated from the co-solvent, glycerol, and etherification products prior to the recycling step or separation step 302 or 502.

The systems for generating glycerol tert-butyl ethers through etherification reactions of glycerol and isobutylene can be any system known in the art configured for performing etherification reactions. For example, and not by way of limitation, the system can include one or more reactors. The reactor can be of various designs known in the art, e.g., a continuously stirred tank reactor (CSTR), a batch reactor, a mechanically stirred tank reactor, a trickle-flow fixed bed reactor, a bubble column reactor (BCR), or any other kind of reactor suitable for gas/liquid reactions. In certain embodiments, the one or more reactors can be configured in parallel. Alternatively or additionally, the one or more reactors can be configured in series. The reactor can be constructed of any suitable materials such as, but not limited to, metals, alloys (including steel), glasses, enamels, ceramics, polymers, or a combination comprising at least one of the foregoing. The reactor can include a reaction vessel enclosing a reaction chamber.

The reaction vessel can be any suitable design or shape such as, but not limited to, tubular, cylindrical, rectangular, dome or bell-shaped. The dimensions size of the reaction vessel and reaction chamber are variable and can depend on the desired reaction type, production capacity, feed volume, and catalyst. For example, and not by way of limitation, the reactor size can be up to 20,000 L (e.g., for commercial reactors). In certain embodiments, the reactor can optionally include a tray on which a catalyst, e.g. a solid acid catalyst, is positioned. The geometries of the reactor and of the overall systems for generating glycerol tert-butyl ethers are adjustable in various ways known to one of ordinary skill in the art. The residence time can be optimized by adjustment of various reactor parameters, e.g., flow rates of inputs into the reactor or outputs out of the reactor.

In certain embodiments, the system can include one or more separators to remove the unreacted isobutylene from the etherification products, co-solvent, or unreacted reactants. In certain embodiments, the separator can function to remove the desired etherification products from the co-solvent or unreacted reactants. In certain embodiments, the system can further include a mixer for combining the reactants, co-solvents or recycled etherification products prior to introduction into the one or more reactors. In certain embodiments, the system can further include one or more transfer lines for transferring the etherification products, co-solvent or unreacted reactants to the mixer or the one or more reactants.

The following Maple is merely illustrative of the presently disclosed subject matter and should not be considered as a limitation in any way.

### EXAMPLE

This Example describes the effect of methanol addition on the formation of isobutylene (B) oligomers (i.e., the oligomerization of isobutylene, which is an undesired secondary reaction) during an etherification reaction. The reaction was performed for 130 minutes +/−7 minutes.

TABLE 1

| | Iso-oligomer formation with respect to initial IB (wt. %) | | |
|---|---|---|---|
| Component | With 20% MeOH | With 10% MeOH | 0% MeOH |
| IB Oligomer | 0.824 | 1.507 | 2.766 |

As shown in Table 1, the addition of methanol led to a reduction in the amount of isobutylene oligomers formed during the esterification reaction. The percentage of isobutylene oligomers that was formed is shown as a percentage of the amount if isobutylene that was initially added to the reaction. As shown in Table 1, with an increase in concentration of methanol, the oligomerization of isobutylene, which leads to di-isobutylene and its isomers, decreased significantly. In the presence of 10% methanol, the oligomerization decreased by 45%, and with 20% methanol, the oligomerization decreased by 70%, as compared to the reaction without methanol.

The claims are further illustrated by the following Embodiments, which are not intended to be limiting.

Embodiment 1. A method for the production of glycerol tert-butyl ether comprising: reacting isobutylene and glycerol in the presence of a catalyst and a co-solvent cum co-reactant to generate a mono-, di-, or tri-glycerol tert-butyl ether, wherein the isobutylene and glycerol are partially miscible.

Embodiment 2. The method of claim 1, wherein the co-solvent cum co-reactant is methanol, ethanol, methyl tert-butyl ether, ethyl tert-butyl ether, or a combination comprising at least one of the foregoing.

Embodiment 3. The method of claim 1, wherein the co-solvent cum co-reactant is methanol.

Embodiment 4. The method of claim 1, wherein the co-solvent cum co-reactant is ethanol.

Embodiment 5. The method of claim 3, further comprising reacting methanol with isobutylene to produce methyl tert-butyl ether.

Embodiment 6. The method of claim 4, further comprising reacting ethanol with isobutylene to produce ethyl tert-butyl ether.

Embodiment 7. The method of claim 1, wherein the catalyst is an acid catalyst.

Embodiment 8. The method of claim 7, wherein the acid catalyst is sulfuric acid, p-toluene sulfonic acid, methanesulfonic acid, or a combination comprising at least one of the foregoing.

Embodiment 9. The method of claim 1, further comprising separating the di- and tri-glycerol tert-butyl from the mono-glycerol tert-butyl ether and recycling the co-solvent cum co-reactant, unreacted glycerol, if any, and mono-glycerol tert-butyl for further generation of a mono-, di-, or tri-glycerol tert-butyl ether product.

Embodiment 10. The method of claim 5, further comprising separating the di- and tri-glycerol tert-butyl and methyl tert-butyl ether from the mono-glycerol tert-butyl ether and recycling the co-solvent cum co-reactant, unreacted glycerol, if any, and mono-glycerol tert-butyl for further generation of a mono-, di-, or tri-glycerol tert-butyl ether product.

Embodiment 11. The method of claim 6, further comprising separating the di- and tri-glycerol tert-butyl and ethyl tert-butyl ether from the mono-glycerol tert-butyl ether and recycling the co-solvent cum co-reactant, unreacted glycerol, if any, and mono-glycerol tert-butyl for further generation of a mono-, di-, or tri-glycerol tert-butyl ether product.

Embodiment 12. A method for the production of glycerol tert-butyl ether comprising: reacting isobutylene and glycerol in the presence of a catalyst and methanol, ethanol, methyl tert-butyl ether, ethyl tert-butyl ether, or a combination comprising at least one of the foregoing as a co-solvent cum co-reactant, to produce a mono-, di-, or tri-glycerol tert-butyl ether, wherein the isobutylene and glycerol are partially miscible.

Embodiment 13. A method for the production of glycerol tert-butyl ether comprising:reacting isobutylene and glycerol in the presence of a catalyst and a co-solvent comprising methanol, methyl tert-butyl ether, and mono-glycerol tert-butyl ether to produce a mono-, di-, or tri-glycerol tert-butyl ether, wherein the isobutylene and glycerol are partially miscible.

Embodiment 14. A method for the production of glycerol tert-butyl ether and methyl tert-butyl ether, the method comprising: reacting isobutylene and glycerol in the presence of an acid catalyst and methanol or ethanol to produce a mono-, di-, or tri-glycerol tert-butyl ether and methyl tert-butyl ether or ethyl tert-butyl ether, respectively, wherein the isobutylene and glycerol are partially miscible.

Embodiment 15. The method of claim 15, wherein the acid catalyst is sulfuric acid, p-toluene, methanesulfonic acid, or a combination comprising at least one of the foregoing.

The compositions and methods articles can alternatively comprise, consist of, or consist essentially of, any appropriate components or steps herein disclosed. The compositions and methods can additionally, or alternatively, be formulated so as to be devoid, or substantially free, of any steps, components, materials, ingredients, adjuvants, or species that are otherwise not necessary to the achievement of the function and/or objectives of the compositions, methods, and articles.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity, and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments.

Unless defined otherwise, technical and scientific terms used herein have the same meaning as is commonly understood by one of skill in the art to which this invention belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Although the presently disclosed subject matter and its advantages have been described in detail, it should be understood that various changes, substitutions, and alterations can be made herein without departing from the spirit and scope of the disclosed subject matter as defined by the appended claims. In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

We claim:

1. A method for the production of glycerol tert-butyl ether comprising:
reacting isobutylene and glycerol in the presence of a catalyst and a co-solvent cum co-reactant to generate a mono-, di-, or tri-glycerol tert-butyl ether,
wherein the isobutylene and glycerol are partially miscible.

2. The method of claim 1, wherein the co-solvent cum co-reactant is methanol, ethanol, methyl tert-butyl ether, ethyl tert-butyl ether, or a combination comprising at least one of the foregoing.

3. The method of claim 1, wherein the co-solvent cum co-reactant is methanol.

4. The method of claim 1, wherein the co-solvent cum co-reactant is ethanol.

5. The method of claim 3, further comprising reacting methanol with isobutylene to produce methyl tert-butyl ether.

6. The method of claim 4, further comprising reacting ethanol with isobutylene to produce ethyl tert-butyl ether.

7. The method of claim 1, wherein the catalyst is an acid catalyst.

8. The method of claim 7, wherein the acid catalyst is sulfuric acid, p-toluene sulfonic acid, methanesulfonic acid, or a combination comprising at least one of the foregoing.

9. The method of claim 1, further comprising separating the di- and tri-glycerol tert-butyl from the mono-glycerol tert-butyl ether and recycling the co-solvent cum co-reactant, unreacted glycerol, if any, and mono-glycerol tert-butyl for further generation of a mono-, di-, or tri-glycerol tert-butyl ether product.

10. The method of claim 5, further comprising separating the di- and tri-glycerol tert-butyl and methyl tert-butyl ether from the mono-glycerol tert-butyl ether and recycling the co-solvent cum co-reactant, unreacted glycerol, if any, and mono-glycerol tert-butyl for further generation of a mono-, di-, or tri-glycerol tert-butyl ether product.

11. The method of claim 6, further comprising separating the di- and tri-glycerol tert-butyl and ethyl tert-butyl ether from the mono-glycerol tert-butyl ether and recycling the co-solvent cum co-reactant, unreacted glycerol, if any, and mono-glycerol tert-butyl for further generation of a mono-, di-, or tri-glycerol tert-butyl ether product.

12. A method for the production of glycerol tert-butyl ether comprising:
reacting isobutylene and glycerol in the presence of a catalyst and methanol, ethanol, methyl tert-butyl ether, ethyl tert-butyl ether, or a combination comprising at least one of the foregoing as a co-solvent cum co-reactant, to produce a mono-, di-, or tri-glycerol tert-butyl ether,
wherein the isobutylene and glycerol are partially miscible.

13. A method for the production of glycerol tert-butyl ether comprising:
reacting isobutylene and glycerol in the presence of a catalyst and a co-solvent comprising methanol, methyl tert-butyl ether, and mono-glycerol tert-butyl ether to produce a mono-, di-, or tri-glycerol tert-butyl ether,
wherein the isobutylene and glycerol are partially miscible.

14. A method for the production of glycerol tert-butyl ether and methyl tert-butyl ether, the method comprising:
reacting isobutylene and glycerol in the presence of an acid catalyst and methanol or ethanol to produce a mono-, di-, or tri-glycerol tert-butyl ether and methyl tert-butyl ether or ethyl tert-butyl ether, respectively,
wherein the isobutylene and glycerol are partially miscible.

15. The method of claim 14, wherein the acid catalyst is sulfuric acid, p-toluene, methanesulfonic acid, or a combination comprising at least one of the foregoing.

16. The method of claim 12, wherein the catalyst is sulfuric acid, p-toluene, methanesulfonic acid, or a combination comprising at least one of the foregoing.

17. The method of claim 13, wherein the catalyst is sulfuric acid, p-toluene, methanesulfonic acid, or a combination comprising at least one of the foregoing.
